(19) United States
(12) Patent Application Publication
(10) Pub. No.: US 2020/0202983 A1
(43) Pub. Date: Jun. 25, 2020

(54) SIMULATION METHOD, SIMULATION APPARATUS, AND PROGRAM

(71) Applicant: SUMITOMO HEAVY INDUSTRIES, LTD., Tokyo (JP)

(72) Inventor: Yuya Matsumura, Kanagawa (JP)

(21) Appl. No.: 16/677,970

(22) Filed: Nov. 8, 2019

(30) Foreign Application Priority Data
Dec. 20, 2018 (JP) 2018-238196
Dec. 20, 2018 (JP) 2018-238197

(51) Int. Cl. G16C 10/00; G06F 17/50

(52) U.S. Cl. CPC G16C 10/00; G06F 17/5009

(57) ABSTRACT

Simulation is performed by using a molecular dynamics method in which a flow field having an inflow/outflow interface is used as an analysis region, and a fluid in the flow field is set as an aggregate of a plurality of particles. An analysis model is defined in which the inflow/outflow interface is divided into plural partitions, a heat bath is coupled to the inflow/outflow interface, and a particle is allowed to move between the heat bath and the analysis region. A relationship between an in-face position of the inflow/outflow interface and a pressure target value is acquired. The heat bath is divided into plural heat bath cells in correspondence with the plural partitions, and a pressure in a heat bath cell is controlled on the basis of the pressure target value in a corresponding partition in a case where a state of the particle is temporally developed.

### RELATED APPLICATIONS

The contents of Japanese Patent Application No. 2018-238196 and Japanese Patent Application No. 2018-238197, on the basis of each of which priority benefits are claimed in an accompanying application data sheet, are in their entirety incorporated herein by reference.

### BACKGROUND

### Technical Field

A certain embodiment of the present invention relates to a simulation method, a simulation apparatus, and a program using a molecular dynamics method or a renormalized molecular dynamics method.

### Description of Related Art

In a case where the temperature of steam decreases at a low pressure stage of a steam turbine, condensation occurs, and thus water droplets are generated. The water droplets collide with a rotor blade, and thus erosion occurs in the rotor blade. In order to suppress the erosion, it is important to understand behaviors of steam and water droplets in the steam turbine. In the related art, in simulation analysis for a flow field of a fluid such as steam, the fluid is handled as a continuum (for example, refer to the related art). In such simulation analysis, it is hard to understand a detailed behavior of a flow field accompanied by a phase change from a gas to a liquid.

A technique has been proposed in which a behavior of a fluid is analyzed by performing simulation analysis according to a molecular dynamics method or a renormalized molecular dynamics method (for example, refer to the related art).

### SUMMARY

According to an aspect of the present invention, there is provided a simulation method of performing simulation by using a molecular dynamics method, in which a flow field having an inflow/outflow interface is used as an analysis region, and a fluid in the flow field is set as an aggregate of a plurality of particles, the simulation method including in an analysis model in which the inflow/outflow interface is divided into a plurality of partitions, a heat bath is coupled to the inflow/outflow interface, and a particle is allowed to move between the heat bath and the analysis region, acquiring a relationship between an in-face position of the inflow/outflow interface and a pressure target value; and controlling a pressure in a heat bath cell on the basis of the pressure target value in a corresponding partition in a case where a state of the particle is temporally developed, the heat bath being divided into a plurality of the heat bath cells in correspondence with the plurality of partitions.

### BRIEF DESCRIPTION OF THE DRAWINGS

FIG. 1 is a block diagram illustrating a simulation apparatus according to an embodiment.

FIG. 2A is a schematic diagram illustrating an example of an analysis model that is an object analyzed according to a simulation method of the embodiment, and FIG. 2B is a graph illustrating an example of a relationship between an in-face position of an inflow/outflow interface and a pressure target value.

FIG. 3 is a flowchart illustrating process procedures executed by a processing unit of the simulation apparatus according to the embodiment.

FIG. 4 is a flowchart illustrating procedures of pressure control in the inflow/outflow interface.

FIG. 5A is a schematic diagram illustrating a first heat bath cell in a state in which particles are added in a case where the number dN is positive, and FIG. 5B is a schematic diagram illustrating a second heat bath in a state in which particles are removed in a case where the number dN is negative.

FIG. 6 is a schematic diagram illustrating an analysis model for simulation that was actually performed.

FIG. 7A is a graph illustrating a pressure target value in the inflow/outflow interface and a computation value of a pressure in a first heat bath when a steady state is reached, and FIG. 7B is a diagram illustrating a position of a particle when the steady state is reached.

FIG. 8 is a perspective view illustrating an analysis model that is an object analyzed according to a simulation method of another embodiment.

FIG. 9 is a flowchart illustrating procedures in which pressure control (step S4 in FIG. 3) is performed.

FIG. 10 is a schematic diagram illustrating an example of an analysis model that is an object analyzed according to a simulation method of still another embodiment.

FIG. 11 is a flowchart illustrating procedures of pressure control in an inflow/outflow interface according to a simulation method of the embodiment illustrated in FIG. 10.

FIG. 12A is a graph illustrating distributions of pressure in a nozzle axis direction, obtained through simulation according to methods of the embodiment and a comparison example, and FIG. 12B is a graph obtained by enlarging a part of FIG. 12A.

### DETAILED DESCRIPTION

In simulation analysis of the related art using a molecular dynamics method, it is hard to handle a system accompanied by inflow and outflow of a fluid. It is desirable to provide a simulation method, a simulation apparatus, and a program for analyzing a system accompanied by inflow and outflow of a fluid by using a molecular dynamics method. In the present specification, a renormalized molecular dynamics method may be said to be a molecular dynamics method in a broad sense. In the present specification, the molecular dynamics method and the renormalized molecular dynamics method will be simply referred to as a “molecular dynamics method”.

Since a heat bath is coupled to an inflow/outflow interface of an analysis region, and a pressure in the heat bath is controlled, simulation can be performed in a state in which a pressure in the inflow/outflow interface is maintained at a target value.

According to another aspect of the present invention, there are provided a simulation apparatus executing the simulation method and a program causing a computer to execute the simulation method.

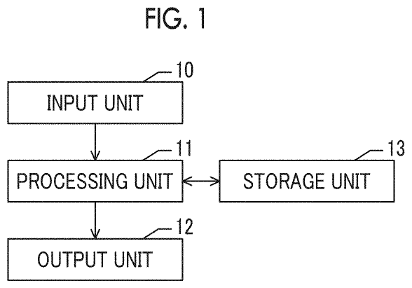

With reference to FIGS. 1 to 5B, a description will be made of a simulation method and a simulation apparatus according to an embodiment.

FIG. 1 is a block diagram illustrating a simulation apparatus according to an embodiment. The simulation apparatus according to the embodiment includes an input unit 10, a processing unit 11, an output unit 12, and a storage unit 13. Simulation conditions or the like are input to the processing unit 11 from the input unit 10. Various commands or the like are input to the input unit 10 from an operator. The input unit 10 includes, for example, a communication device, a removable reading device, a keyboard, and the like.

The processing unit 11 performs simulation using a molecular dynamics method or a renormalized molecular dynamics method (hereinafter, simply referred to as a molecular dynamics method) on the basis of the input simulation conditions and commands. Simulation results are output to the output unit 12. The simulation results include information indicating a particle state of a particle system that is a simulation object, information indicating a temporal change of a physical quantity of the particle system, and the like. The processing unit 11 includes, for example, a computer, and a program for causing the computer to execute simulation using the molecular dynamics method is stored in the storage unit 13. The output unit 12 includes a communication device, a removable writing device, a display, and the like.

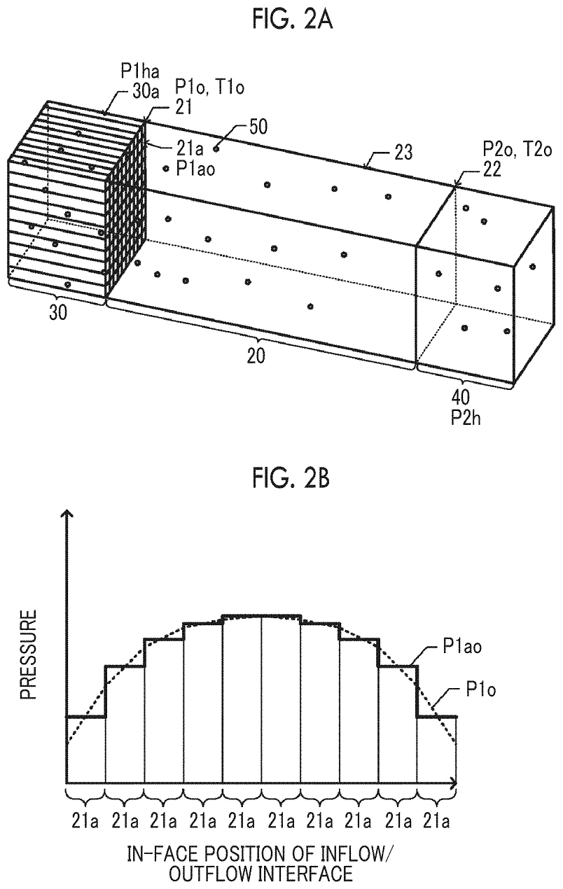

FIG. 2A is a schematic diagram illustrating an example of an analysis model that is an object analyzed according to a simulation method of the embodiment. For example, a square pillar-shaped analysis region 20 having wall surfaces 23 and a pair of inflow/outflow interfaces 21 and 22 is defined. A flow field in which a fluid, for example, water vapor flows into the analysis region 20 from one inflow/outflow interface 21, and the water vapor flows to the outside from the other inflow/outflow interface 22 is formed in the analysis region 20. This fluid is expressed by an aggregate of a plurality of particles, behaviors of the particles are analyzed by using a molecular dynamics method, and thus analysis of the flow field in the analysis region 20 is analyzed. As boundary conditions, target values (a temperature target value T1o and a pressure target value P1o) of a temperature and a pressure at one inflow/outflow interface 21 and target values (a temperature target value T2o and a pressure target value P2o) of a temperature and a pressure at the other inflow/outflow interface 22 are given.

A first heat bath 30 is coupled to the inflow/outflow interface 21, and a second heat bath 40 is coupled to the other inflow/outflow interface 22. A particle 50 moves between the first heat bath 30 and the analysis region 20 through the inflow/outflow interface 21. Similarly, the particle 50 moves between the second heat bath 40 and the analysis region 20 through the inflow/outflow interface 22. Reflectance boundary conditions are applied to surfaces except the inflow/outflow interface 21 among surfaces the first heat bath 30 and surfaces except the inflow/outflow interface 22 among surfaces of the second heat bath 40. The particle 50 that comes into contact with a reflectance boundary is reflected with a velocity component in a face normal direction opposite to a direction when coming into contact. For example, a cyclic boundary condition or a reflectance boundary condition is applied to the wall surfaces 23 of the analysis region 20. If a pressure in the first heat bath 30 is set to be higher than a pressure in the second heat bath 40, a flow field directed from the inflow/outflow interface 21 to the inflow/outflow interface 22 is formed in the analysis region 20.

The inflow/outflow interface 21 is divided into a plurality of partitions 21a. The inflow/outflow interface 21 is divided in a grid shape. The first heat bath 30 is divided into a plurality of first heat bath cells 30a in correspondence with the plurality of partitions 21a. The plurality of first heat bath cells 30a are coupled to the analysis region 20 via the respective corresponding partitions 21a. The two first heat bath cells 30a corresponding to the two partitions 21a adjacent to each other are continued to each other, and an interface between both thereof allows the particle 50 to move between the two first heat bath cells 30a.

FIG. 2B is a graph illustrating an example of a relationship between an in-face position and the pressure target value P1o of the inflow/outflow interface 21. A transverse axis expresses an in-face position of the inflow/outflow interface 21 in a one-dimensional direction, and a longitudinal axis expresses pressure. The pressure target value P1o having a distribution regarding an in-face of the inflow/outflow interface 21 is given as one of the simulation conditions. A pressure target value P1ao is determined for each partition 21a such that the distribution of the pressure target value P1o is reflected. For example, the pressure target value P1o is discretized by a dimension of the partitions 21a, and the discretized value is set as the pressure target value P1ao in the partition 21a.

In a case where the analysis model illustrated in FIG. 2A is analyzed according to a typical molecular dynamics method, the particle 50 in the first heat bath 30 coupled to the inflow/outflow interface 21 on the upstream side of the flow field flows into the analysis region 20, and thus the number of particles decreases with the passage of time. Conversely, the particle 50 flows from the analysis region 20 into the second heat bath 40 coupled to the inflow/outflow interface 22 on the downstream side of the flow field, and thus the number of particles increases with the passage of time. Due to the change of the number of particles, the pressure in the first heat bath 30 decreases with the passage of time, and the pressure in the second heat bath 40 increases with the passage of time. Thus, the pressure in the inflow/outflow interface 21 and the inflow/outflow interface 22 cannot be maintained to be constant. In the embodiment described below, a process of maintaining the pressure in the inflow/outflow interface 21 and the inflow/outflow interface 22 at the constant pressure target values P1o and P2o.

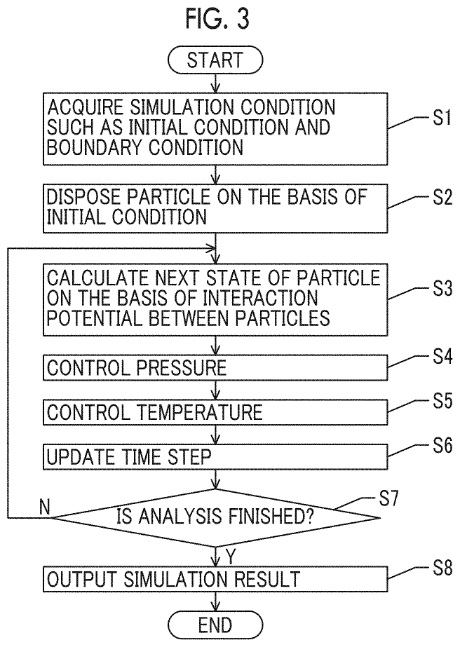

FIG. 3 is a flowchart illustrating process procedures executed by the processing unit 11 (FIG. 1) of the simulation apparatus according to the embodiment.

First, the processing unit 11 acquires simulation conditions such as initial conditions and boundary conditions for simulation that are input to the input unit 10 (step S1). The boundary conditions include shapes and sizes of the analysis region 20, the first heat bath 30, and the second heat bath 40, and the pressure target values P1o and P2o and the temperature target values T1o and T2o in the inflow/outflow interfaces 21 and 22. The initial conditions include information indicating a position and a velocity of the particle 50. The simulation conditions include the mass and a size of the particle 50, information defining interaction potential between particles, and information regarding a time stride at which temporal development is performed. For example, a Leonard-Jones potential may be used as the interaction potential between the particles. The information defining the interaction potential includes, for example, a fitting parameter of the Leonard-Jones potential.

In a case where the simulation conditions are acquired, the processing unit 11 disposes a plurality of particles 50 in the first heat bath 30, the analysis region 20, and the second heat bath 40 on the basis of the initial conditions. A motion equation is solved on the basis of the interaction potential between the particles 50, and thus the next state of the particle 50 is calculated (step S3). Specifically, a position and a velocity of the particle 50 after one time step are calculated.

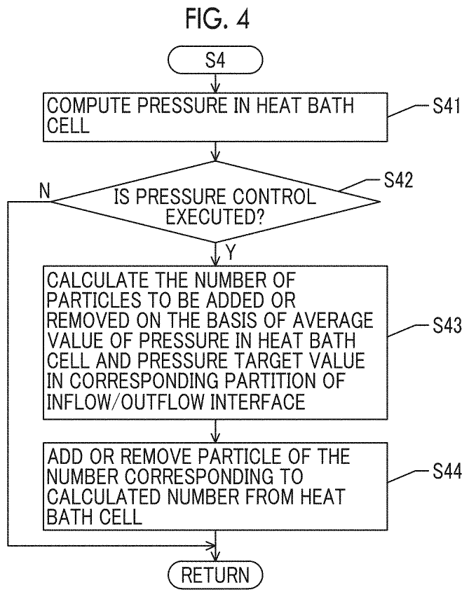

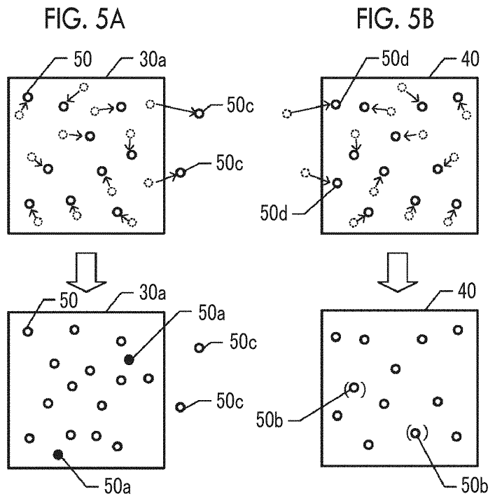

In a case where the next state of the particle 50 is obtained, pressure control for maintaining pressures P1 and P2 in the inflow/outflow interfaces 21 and 22 to be pressure target values is performed (step S4). Details of the pressure control will be described with reference to FIGS. 4, 5A, and 5B. Herein, a summary of the pressure control will be described.

First, a description will be made of pressure control in the inflow/outflow interface 21. A pressure P1ha in the first heat bath cell 30a (FIG. 2) in the latest state (that is, the next state of the particle 50 obtained in step S3) of the particle 50 is computed. The pressure P1ha obtained through the computation is compared with the pressure target value P1ao in the partition 21a. The particle 50 is added to the first heat bath cell 30a or the particle 50 is removed from the first heat bath cell 30a such that the pressure in the first heat bath cell 30a becomes the pressure target value P1ao on the basis of a comparison result.

Next, a description will be made of the pressure control in the inflow/outflow interface 22. A pressure P2h in the second heat bath 40 (FIG. 2) in the latest state of the particle 50 obtained in step S3 is computed. The pressure P2h in the second heat bath 40 is compared with the pressure target value P2o. The particle 50 is added to the second heat bath 40 or the particle 50 is removed from the second heat bath 40 such that the pressure in the second heat bath 40 becomes the pressure target value P2o on the basis of a comparison result.

After the pressure control is performed, temperature control for maintaining temperatures T1 and T2 in the inflow/outflow interfaces 21 and 22 at temperature target values is performed (step S5). Specifically, control is performed such that a temperature of the particle 50 in the first heat bath cell 30a is maintained at the temperature target value T1o in the inflow/outflow interface 21, and a temperature of the particle 50 in the second heat bath 40 is maintained at the temperature target value T2o in the inflow/outflow interface 22. For example, a velocity scaling method may be used for the temperature control.

In a case where the temperature target value T1o in the inflow/outflow interface 21 is not constant in the face thereof and has a distribution, the temperature control is performed for each first heat bath cell 30a.

The pressure control and the temperature control are performed, and then a time step is updated (step S6). Specifically, a state of the particle 50 obtained after performing the pressure control and the temperature control on the next state of the particle 50 calculated in step S3 is set as the current state.

The processes from step S3 to step S6 are repeatedly performed until the analysis is finished (step S7). In a case where the analysis is finished, a simulation result is output to the output unit 12 (FIG. 1) (step S8). The information output to the output unit 12 may include information indicating temporal changes of pressures in the inflow/outflow interfaces 21 and 22 and temporal changes of particle states in the analysis region 20. The temporal change of the pressure in the inflow/outflow interface 21 may be output such that a temporal change of a pressure of each partition 21a can be understood.

Next, with reference to FIGS. 4, 5A, and 5B, a description will be made of the pressure control (step S4 in FIG. 3) in the inflow/outflow interface 21 (FIG. 2). FIG. 4 is a flowchart illustrating procedures of the pressure control in the inflow/outflow interface 21 (FIG. 2). First, a pressure in the first heat bath cell 30a is computed (step S41). The pressure in the first heat bath cell 30a may be computed by using, for example, the virial theorem.

Next, it is determined whether or not the pressure control is to be executed at the present time step (step S42). For example, the pressure control is executed once every hundreds of time steps. In a case where the pressure control is not executed, the pressure control process is finished, and the flow returns to the flowchart illustrated in FIG. 3. In a case where the pressure control is executed, the number of particles to be added to or removed from the first heat bath cell 30a is calculated such that the pressure in the first heat bath cell 30a is maintained at the pressure target value P1ao on the basis of an average value of the pressures P1ha in the first heat bath cell 30a and the pressure target value P1ao in the corresponding partition 21a (step S43). The average value of the pressures P1ha in the first heat bath cell 30a is obtained by averaging the pressures P1ha at a plurality of past time steps obtained in step S41.

Hereinafter, a description will be made of a method of computing the number of particles to be added or removed. If a total number of particles 50 in the first heat bath cell 30a in the latest state calculated in step S3 is indicated by N, the number dN of particles to be added is calculated according to the following equation.

$${ }_{dN} = floor ( \frac{P 1 ao - P 1 ha}{P 1 ha} \times N ) \qquad ( 1 )$$

Here, the floor function is a function for producing an integer by truncating decimal places. Instead of truncating decimal places, an integer may be produced by rounding up decimal places, and may be produced by rounding off the first decimal place.

Particles of the number corresponding to the calculated number dN are added to or removed from the first heat bath cell 30a (step S44). Specifically, in a case where the number dN is positive, the particles 50 are added, in a case where the number dN is negative, the particles 50 are removed, and, in a case where the number dN is 0, the particles 50 are neither added nor removed.

Next, a description will be made of the pressure control (step S4 in FIG. 3) in the inflow/outflow interface 22 (FIG. 2). In the inflow/outflow interface 22, a pressure in the second heat bath 40 is controlled by using an average value of the pressures P2h in the second heat bath 40 and the pressure target value P2o. In other words, the particles 50 are added to or removed from the second heat bath 40 in the same manner as for the first heat bath cell 30a.

With respect to the second heat bath 40, if a total number of particles 50 in the second heat bath 40 is indicated by N, the number dN of particles to be added is calculated according to the following equation.

$${ }_{dN} = floor ( \frac{P 2 o - P 2 h}{P 2 h} \times N ) \qquad ( 2 )$$

FIG. 5A is a schematic diagram of the first heat bath cell 30a illustrating a state in which the particles 50 are added in a case where the number dN is positive. In FIG. 5A, the last position of the particle 50 is indicated by a dashed line, and a position of the particle 50 in the latest state (the next state obtained in step S3) is indicated by a solid line. In a case where transition occurs from the last state to the latest state, two particles 50c flow out of the first heat bath cell 30a. In the latest state, the number of particles in the first heat bath cell 30a is reduced, and thus a pressure therein decreases. Thus, a relationship of P1ao>P1ha is established, and the number dN is positive. As an example, in a case where the number dN is 2, as illustrated in a lower part of FIG. 5A, two particles 50a are added to the first heat bath cell 30a.

If a distance between the added new particle 50a and the particle 50 that is already present is too short, large repulsive force due to the Leonardo-Johns potential act on both of the particles. As a result, the particles are rapidly accelerated, and there is a probability that calculation may fail. In a case where repulsive force acting between the added new particle 50a and the particle 50 that is already present exceeds a predetermined allowable upper limit value, the added new particles 50a are redisposed.

FIG. 5B is a schematic diagram of the second heat bath 40 illustrating a state in which the particles 50 are removed in a case where the number dN is negative. In FIG. 5B, the last position of the particle 50 is indicated by a dashed line, and a position of the particle 50 in the latest state is indicated by a solid line. In a case where transition occurs from the last state to the latest state, two particles 50d flow into the second heat bath 40. In the latest state, the number of particles in the second heat bath 40 increases, and thus a pressure therein increases. Thus, a relationship of P2o<P2h is established, and the number dN is negative. As an example, in a case where the number dN is −2, as illustrated in a lower part of FIG. 5B, two particles 50b are removed from the second heat bath 40.

Next, a description will be made of excellent effects of the embodiment. In the embodiment, the pressure P1ha in the first heat bath cell 30a is maintained at the pressure target value P1ao in the corresponding partition 21a of the inflow/outflow interface 21, and thus a pressure boundary condition in the inflow/outflow interface 21 is satisfied. Similarly, a pressure boundary condition in the other inflow/outflow interface 22 is satisfied. As mentioned above, simulation can be performed in a state in which the pressure boundary condition is satisfied.

In the embodiment, since the inflow/outflow interface 21 is divided into a plurality of partitions 21a, simulation can be performed in a state in which a pressure boundary condition is satisfied even in a case where a pressure target value is not uniform in the face of the inflow/outflow interface 21.

Next, a modification example of the embodiment will be described. In the embodiment, the inflow/outflow interface 21 is divided into a plurality of partitions 21a, and, with respect to the other inflow/outflow interface 22, the pressure target value P2o is assumed to be uniform in the face thereof. In a case where the pressure target value P2o in the inflow/outflow interface 22 is not uniform, the inflow/outflow interface 22 may also be divided into a plurality of partitions, and a pressure target value may be set for each partition.

In the embodiment, simulation is performed by applying a reflectance boundary condition or a cyclic boundary condition to the wall surfaces 23 (FIG. 2A) of the analysis region 20, but a pressure boundary condition may be set for the wall surfaces 23. In this case, a heat bath may be coupled to the wall surfaces 23. In a case where a pressure is not uniform in a face, the wall surface 23 may be divided into a plurality of partitions.

In the embodiment, detailed description of the temperature control has not been made, but, in a case where a temperature boundary condition in which a temperature distribution occurs in an in-face direction of the inflow/outflow interface 21 is applied, the temperature control may be performed on each partition 21a of the inflow/outflow interface 21 and each first heat bath cell 30a.

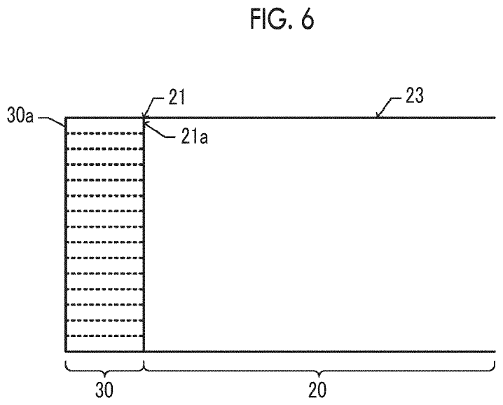

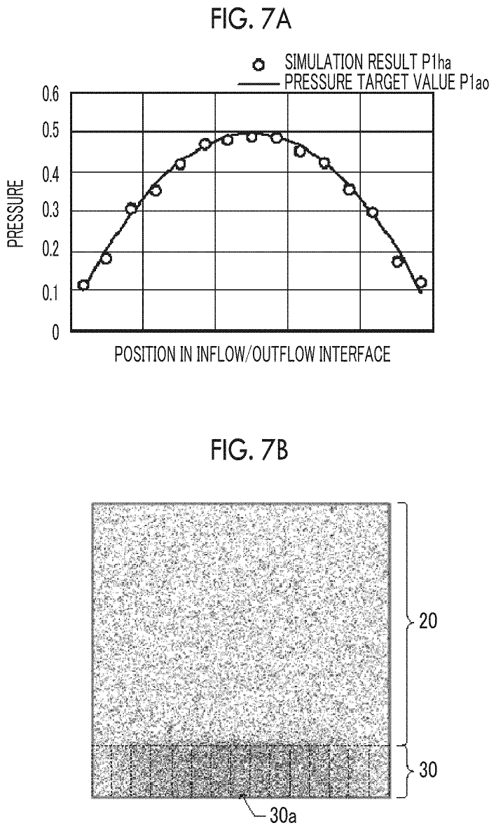

Next, with reference to FIGS. 6 to 7B, a description will be made of simulation performed to check the effects of the embodiment.

FIG. 6 is a schematic diagram illustrating an analysis model for simulation that was actually performed. The first heat bath 30 was coupled to the inflow/outflow interface 21 of the analysis region 20. The analysis region 20 was open on an opposite side to the inflow/outflow interface 21. The inflow/outflow interface 21 was equally divided into fifteen parts in one direction to thus be defined as fifteen partitions 21a. The first heat bath 30 was also equally divided into fifteen parts in correspondence with the partitions 21a to thus be defined as fifteen first heat bath cells 30a.

FIG. 7A is a graph illustrating the pressure target value P1ao in the partition 21a of the inflow/outflow interface 21 and a computation value of a pressure in the first heat bath 30 when a steady state is reached. A transverse axis of FIG. 7A expresses an in-face position of the inflow/outflow interface 21. Both ends of the transverse axis correspond to both ends of the inflow/outflow interface 21. A longitudinal axis expresses pressure as a dimensionless quantity. A solid line illustrated in FIG. 7A indicates the pressure target value P1ao given as a simulation condition, and a circular mark indicates the pressure P1ha in the first heat bath cell 30a obtained through simulation. A distribution was given to the pressure target value P1ao such that a central portion is higher than both end portions. It can be seen that the pressure P1ha in the first heat bath cell 30a has a distribution in which the pressure target value P1ao is reflected.

FIG. 7B is a diagram illustrating a position of a particle when a steady state is reached. It is checked that a particle density in the plurality of first heat bath cells 30a gradually decreases toward the first heat bath cells 30a at both ends from the central first heat bath cell 30a. It can be seen that a distribution of the pressure target value P1ao in the partition 21a is reflected in a distribution of an actual particle density.

It is checked from the simulation results illustrated in FIGS. 6 to 7B that the pressure boundary condition given as a simulation condition is maintained when a plurality of particle states are temporally developed.

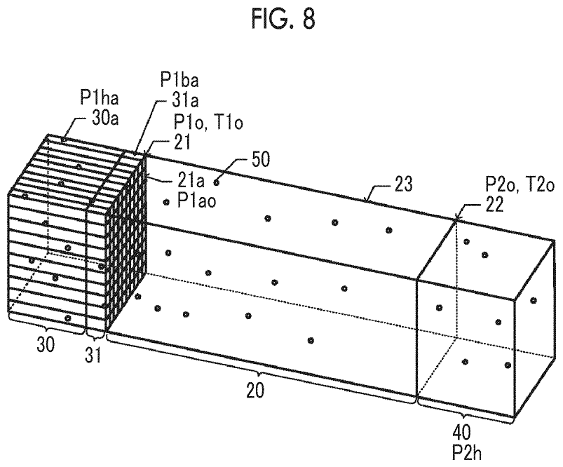

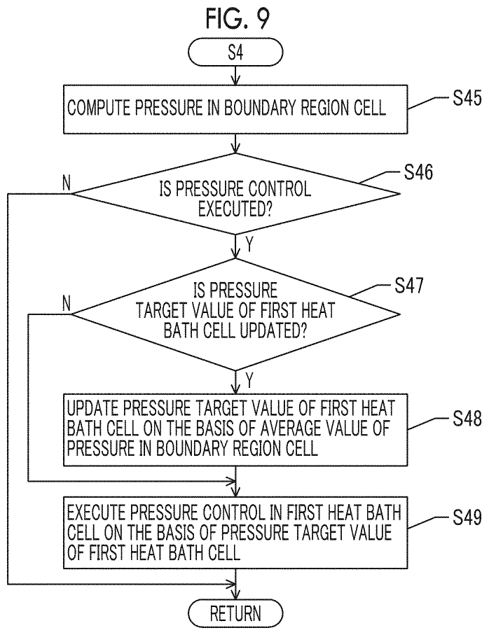

Next, with reference to FIGS. 8 and 9, another embodiment will be described. Hereinafter, description of configurations common to those of the embodiment illustrated in FIGS. 1 to 5B will be omitted.

FIG. 8 is a perspective view illustrating an analysis model that is an object analyzed according to a simulation method of the present embodiment. In the embodiment illustrated in FIG. 2A, the first heat bath 30 is in direct contact with the analysis region 20, but, in the present embodiment, the first heat bath 30 is coupled to the analysis region 20 via a boundary region 31. The boundary region 31 is also divided into a plurality of boundary region cells 31a in correspondence with the plurality of partitions 21a of the inflow/outflow interface 21.

In the present embodiment, the pressure P1ha in the first heat bath cell 30a is controlled such that a pressure in the boundary region cell 31a is maintained at the pressure target value P1ao of the corresponding partition 21a. A particle is not added to or removed from the boundary region cell 31a.

FIG. 9 is a flowchart illustrating procedures for performing pressure control (step S4 in FIG. 3). First, pressures in the plurality of boundary region cells 31a are computed (step S45). The pressures in the boundary region cells 31a may be computed by using, for example, the virial theorem.

Next, it is determined whether or not the pressure control is to be executed at the present time step (step S46). For example, the pressure control is executed once every hundreds of time steps. In a case where the pressure control is not executed, the pressure control process is finished, and the flow returns to the flowchart illustrated in FIG. 3. In a case where the pressure control is executed, it is determined whether or not a pressure target value P1hao of the first heat bath cell 30a is to be updated (step S47). The pressure target value P1hao is updated once, for example, whenever the pressure control is performed ten to hundred times.

In a case where the pressure target value P1hao is not to be updated, the pressure in the first heat bath cell 30a is controlled on the basis of the pressure target value P1hao of the first heat bath cell 30a at the present time (step S49). The pressure target value P1ao in the corresponding partition 21a of the inflow/outflow interface 21 is used as an initial value of the pressure target value P1hao. In a case where the pressure target value P1hao is to be updated, the pressure target value P1hao of the first heat bath cell 30a is updated on the basis of an average value of pressures P1ba in the boundary region cell 31a (step S48). The average value of the pressures P1ba in the boundary region cell 31a is obtained by averaging the pressures P1ba at a plurality of past time steps obtained in step S41. The pressure target value P1hao of the first heat bath cell 30a is determined on the basis of the average value of the pressures P1ba of the boundary region cell 31a, the pressure target value P1ao in the partition 21a of the inflow/outflow interface 21, and the pressure P1ha of the first heat bath cell 30a in the latest state. For example, the pressure target value P1hao is undated on the basis of the following equation.

$$P 1 hao = P 1 hao \cdot \frac{P 1 ao}{P 1 ba} \qquad ( 3 )$$

In Equation (3), P1hao of the left side is a pressure target value after being updated, and P1hao of the right side is a pressure target value at the present time (before being updated). In other words, the pressure target value P1hao is updated on the basis of a ratio between the pressure target value P1ao in the partition 21a of the inflow/outflow interface 21 and the average value of the pressures P1ba of the boundary region cell 31a, and the pressure target value P1hao of the first heat bath cell 30a at the present time.

The pressure target value P1hao is updated, and then the pressure in the first heat bath cell 30a is controlled on the basis of the pressure target value P1hao of the first heat bath cell 30a after being updated (step S49). For example, if the pressure P1ba in the boundary region cell 31a is lower than the pressure target value P1ao, the pressure target value P1hao of the first heat bath cell 30a is made higher than the pressure P1ha in the first heat bath cell 30a in the latest state according to a difference therebetween. Conversely, if the pressure P1ba in the boundary region cell 31a is higher than the pressure target value P1ao, the pressure target value P1hao of the first heat bath cell 30a is made lower than the pressure P1ha in the first heat bath cell 30a in the latest state according to a difference therebetween. As mentioned above, the pressure target value P1hao of the first heat bath cell 30a is corrected at a constant interval.

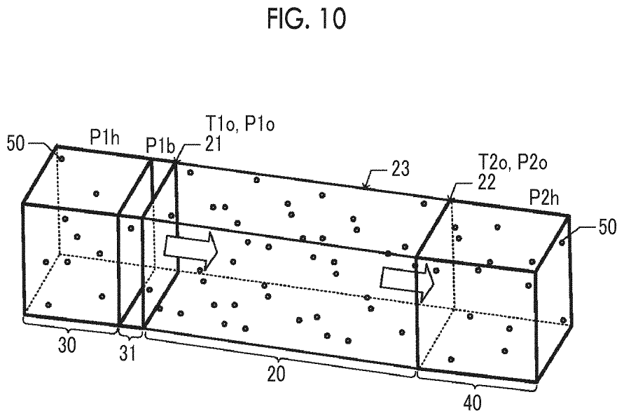

Next, a description will be made of excellent effects of the present embodiment. According to various simulation tests performed by the present inventor, it has found that, if the first heat bath cell 30a to which the particle 50 is added in order to adjust pressure is directly coupled to the analysis region 20, a situation may occur in which pressure discontinuously changes in an interface between both thereof depending on simulation conditions. If the pressure in the interface between both thereof discontinuously changes, even though a pressure in the first heat bath cell 30a is maintained at the pressure target value P1ao, a pressure in a minute region of the analysis region 20 side when viewed from the inflow/outflow interface 21 is deviated from the pressure target value P1ao. The deviation of a pressure will be described later in an embodiment illustrated in FIGS. 10 to 12B.

In the embodiment illustrated in FIGS. 8 and 9, the particle 50 is not added to or removed from the boundary region cell 31a. Thus, a discontinuous change of a pressure in the inflow/outflow interface 21 between the boundary region cell 31a and the analysis region 20 does not occur. The pressure P1ba in the boundary region cell 31a is maintained at the pressure target value P1ao in the partition 21a of the inflow/outflow interface 21. Thus, even though a pressure discontinuously changes in an interface between the first heat bath cell 30a to which the particle 50 is added and the boundary region cell 31a to which the particle is not added, a pressure in a minute region of the analysis region 20 side when viewed from the inflow/outflow interface 21 can be maintained at the pressure target value P1ao.

As described above, the present embodiment is applied to a non-equilibrium molecular dynamics method accompanied by a pressure boundary condition, and thus the pressure boundary condition can be reflected in simulation results with high accuracy.

Next, a description will be made of a modification example of the embodiment illustrated in FIGS. 8 and 9. A cycle of executing the process (step S48 in FIG. 9) of updating the pressure target value P1ho and a cycle of executing pressure control (step S49 in FIG. 9) for the first heat bath 30 may be set to any cycles. If a cycle of executing such a process is too short, a computation load increases. Conversely, if a cycle of executing the process is too long, a difference between a pressure in the inflow/outflow interface 21 (FIG. 8) in which a pressure boundary condition for the analysis region 20 is set and the pressure target value P1o increases. A cycle of executing the process is preferably set such that a difference between a pressure in the inflow/outflow interface 21 in which a pressure boundary condition is set and the pressure target value P1o is included in an allowable range.

Next, with reference to FIGS. 10 to 12B, a description will be made of still another embodiment. Hereinafter, description of configurations common to those of the embodiment illustrated in FIGS. 1 to 5B and the embodiment illustrated in FIGS. 8 and 9 will be omitted.

FIG. 10 is a schematic diagram illustrating an example of an analysis model that is an object analyzed according to a simulation method of the present embodiment. In the embodiment illustrated in FIG. 8, the inflow/outflow interface 21 is divided into a plurality of partitions 21a, and, in correspondence therewith, the first heat bath 30 is divided into a plurality of first heat bath cells 30a, and the boundary region 31 is divided into a plurality of boundary region cells 31a. In contrast, in the present embodiment, the inflow/outflow interface 21 is not divided into a plurality of partitions, and neither of the first heat bath 30 and the boundary region 31 are divided into a plurality of cells. The pressure target value P1o and the temperature target value T1o in the inflow/outflow interface 21 are constant in a face thereof.

The particle 50 is allowed to move between the first heat bath 30 and the boundary region 31 and between the boundary region 31 and the analysis region 20.

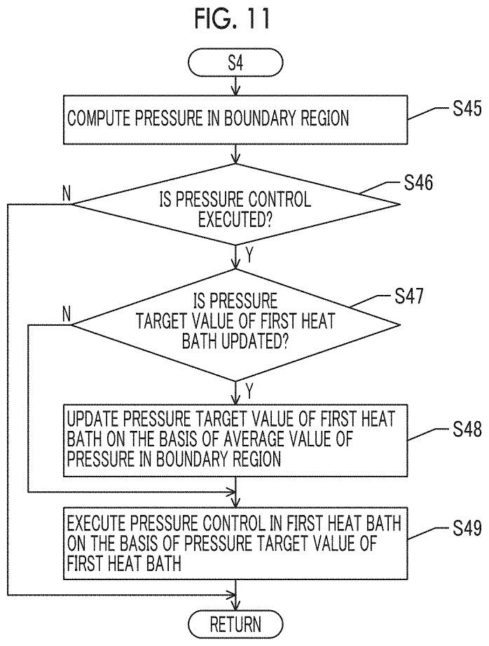

Next, with reference to FIG. 11, a description will be made of pressure control (step S4 in FIG. 3) in the inflow/outflow interface 21 (FIG. 10).

FIG. 11 is a flowchart illustrating procedures for pressure control in the inflow/outflow interface 21 (FIG. 10). Hereinafter, a description will be made through comparison with the flowchart of FIG. 9. In the flowchart of FIG. 11, various computations performed on the first heat bath cell 30a and each boundary region cell 31a in the flowchart of FIG. 9 are performed on the first heat bath 30 and the boundary region 31. Each step in the flowchart of FIG. 11 is given the same reference numeral as the reference numeral added to a corresponding step in the flowchart of FIG. 9.

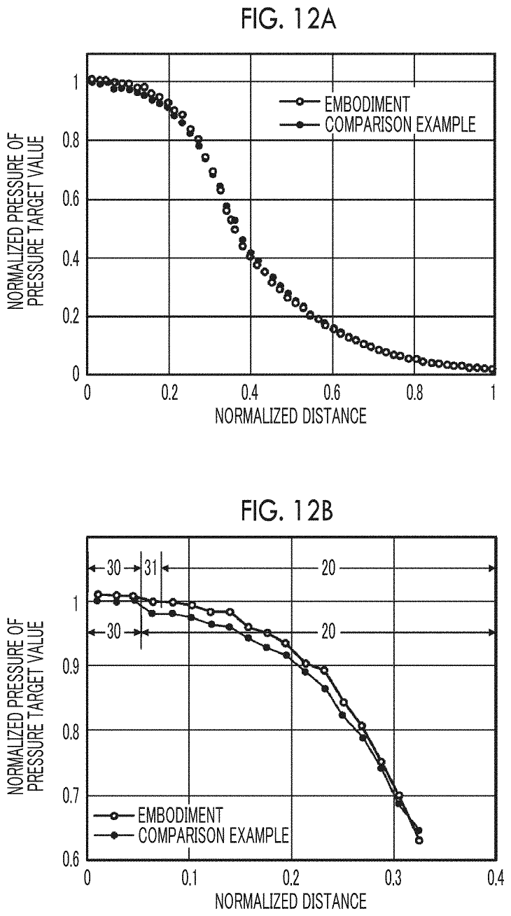

Next, with reference to FIGS. 12A and 12B, a description will be made of simulation performed to check excellent effects of the present embodiment and results thereof.

A pressure distribution of a fluid flowing through a Laval nozzle (convergent-divergent nozzle) was obtained through simulation according to the method of the present embodiment and a method of a comparison example. In the method according to the present embodiment, an inlet of the Laval nozzle was coupled to the first heat bath 30 via the boundary region 31 illustrated in FIG. 10. In the method according to the comparison example, the first heat bath 30 was directly coupled to the inlet of the Laval nozzle. An outlet of the Laval nozzle was open.

FIG. 12A is a graph illustrating distributions of pressure regarding a nozzle axis direction, obtained by performing simulation according to the methods of the embodiment and the comparison example. FIG. 12B is a graph obtained by enlarging a part of FIG. 12A. A transverse axis expresses a normalized distance of a distance from the end face of the first heat bath 30 to the outlet of the Laval nozzle, and a longitudinal axis expresses a normalized pressure of the pressure target value P1o in the inlet. The left end of the transverse axis corresponds to the end face of the first heat bath 30, and the right end thereof corresponds to the outlet of the Laval nozzle. A hollow circular mark and a solid circular mark respectively indicate simulation results according to the embodiment and the comparison example.

As illustrated in FIG. 12A, the pressure decreases toward the outlet from the inlet. The three hollow circular marks and the three solid circular marks from the left end illustrated in FIG. 12B indicate pressures in the first heat bath 30, and the fourth hollow circular mark from the left end indicates a pressure in the boundary region 31. The fifth and subsequent hollow circular marks from the left end, and the fourth and subsequent solid circular marks from the left end indicate pressures in the analysis region 20.

In the comparison example, the normalized pressure in the left end of the analysis region 20 is lower than 1. In other words, the pressure in the left end of the analysis region 20 is not maintained at the pressure target value P1o. In contrast, in the embodiment, the pressure in the first heat bath 30 is controlled such that the normalized pressure in the boundary region 31 is 1, and thus the normalized pressure in the boundary region 31 is maintained at about 1. A change in a pressure in a boundary between the boundary region 31 and the analysis region 20 is gentle, and thus the normalized pressure in the left end of the analysis region 20 is maintained at about 1. In other words, the pressure in the left end of the analysis region 20 is maintained at the pressure target value P1o.

It has been confirmed from the simulation tests that a pressure in a face in which a pressure boundary condition for the analysis region 20 is defined can be maintained at a pressure target value.

Next, a modification example of the embodiment will be described. In the embodiment, the pressure boundary conditions are applied to the inflow/outflow interfaces 21 and 22 (FIG. 2) at both ends of the square pillar-shaped analysis region 20, but pressure boundary conditions may be applied to other faces. In the embodiment, the boundary region 31 is coupled to the inflow/outflow interface 21 on the upstream side of the flow field, but the boundary region 31 may be coupled to the inflow/outflow interface 22 on the downstream side.

In the embodiment, the analysis region 20 has a square pillar shape, but may have any shape. A rigid body which changes its pose or moves through interaction with a fluid may be disposed.

In the embodiment, the Leonard-Jones potential is applied as an interaction potential between particles, but other potentials, for example, a Morse potential may be applied.

The respective embodiments are only examples, and partial replacement of or combination with configurations described in different embodiments may occur. The same advantageous effects achieved by the same configuration of a plurality of embodiments are not sequentially described every embodiment. The present invention is not limited to the embodiments. For example, it is obvious to a person skilled in the art that various changes, modifications, and combinations may occur.

It should be understood that the invention is not limited to the above-described embodiment, but may be modified into various forms on the basis of the spirit of the invention. Additionally, the modifications are included in the scope of the invention.

What is claimed is:

1. A simulation method of performing simulation by using a molecular dynamics method, in which a flow field having an inflow/outflow interface is used as an analysis region, and a fluid in the flow field is set as an aggregate of a plurality of particles, the simulation method comprising:
in an analysis model in which the inflow/outflow interface is divided into a plurality of partitions, a heat bath is coupled to the inflow/outflow interface, and a particle is allowed to move between the heat bath and the analysis region,
acquiring a relationship between an in-face position of the inflow/outflow interface and a pressure target value; and
controlling a pressure in a heat bath cell on the basis of the pressure target value in a corresponding partition in a case where a state of the particle is temporally developed, the heat bath being divided into a plurality of the heat bath cells in correspondence with the plurality of partitions.

2. The simulation method according to claim 1,
wherein two heat bath cells corresponding to two partitions adjacent to each other are in contact with each other via an interface, and the particle is allowed to move between the two heat bath cells.

3. The simulation method according to claim 1,
wherein the pressure in the heat bath cell is controlled by adding the particle to the heat bath cell or removing the particle from the heat bath cell.

4. The simulation method according to claim 1,
wherein the analysis model includes a boundary region coupling the heat bath to the inflow/outflow interface, and allows the particle to move between the heat bath and the boundary region and between the boundary region and the analysis region,
wherein the boundary region is divided into a plurality of boundary region cells in correspondence with the plurality of partitions, and
wherein, in a case where the state of the particle is temporally developed, the pressure in the heat bath cell is controlled such that a pressure in the boundary region cell is maintained at the pressure target value in the corresponding partition when the pressure in the heat bath cell is controlled on the basis of the pressure target value in the corresponding partition.

5. A simulation apparatus performing simulation by using a molecular dynamics method, in which a flow field having an inflow/outflow interface is used as an analysis region, and a fluid in the flow field is set as an aggregate of a plurality of particles, the simulation apparatus comprising:
an input unit to which information for dividing the inflow/outflow interface into a plurality of partitions and a relationship between an in-face position of the inflow/outflow interface and a pressure target value are input; and
a processing unit that uses an analysis model in which a heat bath is coupled to the inflow/outflow interface, and a particle is allowed to move between the heat bath and the analysis region, divides the heat bath into a plurality of the heat bath cells in correspondence with the plurality of partitions, and controls a pressure in a heat bath cell on the basis of the pressure target value in a corresponding partition in a case where a state of the particle is temporally developed.

6. The simulation apparatus according to claim 5,
wherein two heat bath cells corresponding to two partitions adjacent to each other are in contact with each other via an interface, and
wherein the processing unit allows the particle to move between the two heat bath cells and thus temporally develops the state of the particle.

7. The simulation apparatus according to claim 5,
wherein the pressure in the heat bath cell is controlled by adding the particle to the heat bath cell or removing the particle from the heat bath cell.

8. The simulation apparatus according to claim 5,
wherein the analysis model includes a boundary region coupling the heat bath to the inflow/outflow interface, and allows the particle to move between the heat bath and the boundary region and between the boundary region and the analysis region,
wherein the processing unit divides the boundary region into a plurality of boundary region cells in correspondence with the plurality of partitions, and
wherein, in a case where the state of the particle is temporally developed, the processing unit controls the pressure in the heat bath cell such that a pressure in the boundary region cell is maintained at the pressure target value in the corresponding partition when the pressure in the heat bath cell is controlled on the basis of the pressure target value in the corresponding partition.

9. A simulation method of performing simulation by using a molecular dynamics method, in which a flow field having an inflow/outflow interface is used as an analysis region, and a fluid in the flow field is set as an aggregate of a plurality of particles, the simulation method comprising:
in an analysis model in which a heat bath is coupled to the inflow/outflow interface via a boundary region, and a particle is allowed to move between the heat bath and the boundary region and between the boundary region and the analysis region,
acquiring a pressure target value in the inflow/outflow interface; and
controlling a pressure in the heat bath such that a pressure in the boundary region is maintained at the pressure target value when a state of the particle is temporally developed.

10. The simulation method according to claim 9,
wherein the pressure in the heat bath is controlled by adding the particle to the heat bath or removing the particle from the heat bath.

11. The simulation method according to claim 9, further comprising:
acquiring a temperature target value in the inflow/outflow interface; and
controlling a temperature in the heat bath such that the temperature in the heat bath is maintained at the temperature target value when the state of the particle is temporally developed.

12. A simulation apparatus performing simulation by using a molecular dynamics method, in which a flow field having an inflow/outflow interface is used as an analysis region, and a fluid in the flow field is set as an aggregate of a plurality of particles, the simulation apparatus comprising:
an input unit to which a pressure target value in the inflow/outflow interface is input; and
a processing unit that uses an analysis model in which a heat bath is coupled to the inflow/outflow interface via a boundary region, and a particle is allowed to move between the heat bath and the boundary region and between the boundary region and the analysis region, and temporally develops a state of the particle, and controls a pressure in the heat bath such that a pressure in the boundary region is maintained at the pressure target value when the state of the particle is temporally developed.

13. The simulation apparatus according to claim 12,
wherein the pressure in the heat bath is controlled by adding the particle to the heat bath or removing the particle from the heat bath.

14. The simulation apparatus according to claim 12,
wherein a temperature target value in the inflow/outflow interface is further input to the input unit, and
wherein the processing unit controls a temperature in the heat bath such that the temperature in the heat bath is maintained at the temperature target value when the state of the particle is temporally developed.
